(19) United States
(12) Patent Application Publication
(10) Pub. No.: US 2024/0390597 A1
(43) Pub. Date: Nov. 28, 2024

(54) Measurement System for a Drug Delivery Device, Drug Delivery Device with Such a Measurement System and Method for Measuring the Dose Dispensed and/or Dose set of a drug delivery device

(71) Applicant: Sanofi, PARIS (FR)

(72) Inventors: Hester Jane Corne, Warwick, Warwickshire (GB); Paul Richard Draper, Warwick, Warwickshire (GB); Anthony Paul Morris, Warwick, Warwickshire (GB); Ronald Smith, Salir, Loule (PT)

(21) Appl. No.: 18/694,124

(22) PCT Filed: Sep. 22, 2022
(86) PCT No.: PCT/EP2022/076299

§ 371 Date: Mar. 21, 2024

(30) Foreign Application Priority Data
Sep. 24, 2021 (EP) 21315173.1

(51) Int. Cl. A61M 5/315; G16H 20/17

(52) U.S. Cl. CPC A61M 5/31568; A61M 5/31553; G16H 20/17; A61M 2205/3327; A61M 2205/50

(57) ABSTRACT

Measurement system for a drug delivery device, drug delivery device with such a measurement system and method for measuring the dose dispensed and/or dose set of a drug delivery device. Proposed is a measurement system for a drug delivery device, a drug delivery device comprising such a system and a related method, the measurement system comprising—a dose control member configured to move in accordance with a dose dispense operation and/or a dose set operation, e.g., in relation to a housing of the drug delivery device,—a sensor unit for observing a position of the dose control member intermittently, and configured to generate signals, each corresponding to one of at least four different data values, depending on a position and/or orientation of the dose control member, and—an electronic processing unit configured to receive the signals of the sensor unit, to adapt a dose-related count depending on the received signals and to determine whether two consecutive of the received signals correspond to data values that are neighbors in a predetermined repeating sequence of the different data values.

### BACKGROUND

Drug delivery devices, especially needle-based and/or pen-type injection devices, are known to have mechanisms to measure the amount of drug or medicament dispensed from the device.

WO 2019/101962 A1 relates to a medicament injection device with a rotary encoder.

### SUMMARY

It is an object of the present disclosure to provide an improved measurement system, e.g., a measurement system with increased reliability and/or accuracy of the measurement, particularly the measurement of the size of the dose dispensed in a dispensing operation of the drug delivery device.

This object is achieved by the subject-matter of the independent claim. Advantageous embodiments and refinements are subject to dependent claims.

In the present disclosure, a measurement system (electronic measurement system) for a drug delivery device is proposed, comprising a dose control member configured to move in accordance with a dose dispense operation and/or a dose set operation, e.g., in relation to a housing of the drug delivery device; the measurement system further comprising a sensor unit for observing a position of the dose control member in relation to the housing intermittently, and configured to generate signals, each corresponding to one of at least four different data values, depending on a position and/or orientation of the dose control member; and the measurement system further comprising an electronic processing unit configured to receive the signals of the sensor unit, to adapt a dose-related count depending on the received signals and to determine whether two consecutive of the received signals correspond to data values that are neighbors in a predetermined repeating sequence of the different data values.

The dose control member “moving in accordance with a dose dispense operation” may herein mean that an amount of movement, e.g., a length of a linear movement and/or an angle of rotation, of the dose control member is proportional to an amount of drug dispensed during the drug dispense operation. The drug delivery device may comprise a drive unit comprising a converter unit. The drive unit may comprise an energy store, e.g., to drive a dispense operation, or be user driven or manually driven. The converter unit may be configured to convert energy provided by a user and/or from the energy store into pressure acting on a medicament and/or medicament container in the drug delivery device to force the medicament out of the drug delivery device (dispense it). The converter unit may comprise a plunger acting onto a syringe. Alternatively, the converter unit may comprise e.g., a squeezer acting on a medicament tube or bag. The dose control member may be an integral part of the drive unit, e.g., essential for the function of the dose dispense operation. Alternatively, the dose control member may be coupled, e.g., mechanically and/or magnetically, to at least one moving part of the drive unit at least during the dose dispense operation.

The dose control member “moving in accordance with a dose set operation” may herein mean that an amount of movement, e.g., a length of a linear movement and/or an angle of rotation, of the dose control member is proportional to the size of a dose set. The drug delivery device may comprise a dose dial unit for selecting an amount of medicament in the dose set operation. The drive unit may be configured to dispense the amount of medicament selected in the dose set operation in the dose dispense operation. The dose control member may be an integral part of the dose dial unit, e.g., essential for the function of the dose set operation. Alternatively, the dose dial member may be coupled, e.g., mechanically and/or magnetically, to at least one moving part of the drive unit at least during the dose dispense operation.

Depending on the embodiment, the measurement system may comprise a single dose control member and a single sensor unit to observe that dose control member for observing a movement of the dose control member either during a dose set operation or during a dose dispense operation. Alternatively, the same dose control member may be coupled, e.g., via a clutch mechanism, to the dial unit at least during the dose set operation and may be coupled, e.g., via the clutch mechanism, to the drive unit at least during the dose dispense operation. As a further alternative the measurement system may comprise two dose control members, one moving according to the dose set operation and one moving according to the dose dispense operation. When both operations are observed, differences between the set dose and the dispensed dose may be detected.

A “position” of the dose control member may refer to an angular position and/or a location of the dose control member. The drug delivery device may comprise a housing in relation to which the position of the dose control member is observed. The housing may comprise a medicament container or may be configured to hold a medicament container in place in relation to the housing. A position “in relation to the housing” may therefore refer to a position in relation to a medicament container in the drug delivery device, when such a container is in place (installed).

A member of a device being “configured” to perform a specific task may refer to the member performing that task in at least one state of operation of the device. The member may be specially formed, programmed and/or arranged to allow for the performance of the task.

The sensor unit observing “intermittently” may herein mean that the sensor unit is not observing continuously, e.g., to reduce power consumption. The sensor unit may be configured to activate only at specific times. The sensor unit may be configured to switch between a first state in which it does not observe at all and a second state in which the sensor unit observes in predefined intervals, e.g., in regular intervals. The sensor unit may be configured to observe with a specific sample rate, e.g., at least in the second state. The electronic processing unit may be configured to activate and/or deactivate the sensor unit at specific times. The electronic processing unit may be configured to set the specific sample rate at which the sensor unit observes. The sensor unit being inactive (being not active) may herein mean that at least one essential component of the sensor unit, e.g., an amplifier circuit and/or a probe circuit, is not powered and/or that an evaluation circuit of the sensor unit or the electronic processing unit is deactivated or disregards (does not evaluate) signals from the sensor elements.

The sensor unit may comprise at least one sensor element configured to observe a specific physical property of the observed element (dose control member), e.g., reflectivity, magnetization, electric polarization (e.g., ferroelectricity), distance, color, electrical conductivity, or a combination of physical properties. The sensor element may be an electronic sensor element. The sensor element may be configured to change at least one electrical property, e.g., an electrical current, an electrical resistance and/or an electrical voltage, in dependence of the value of the observed physical property. The sensor's elements may be digital or analogue sensors. Digital sensors may have two states that can be distinguished (i.e., a binary 1, and a binary 0, or white and black, or some other combination of two states). The sensor element(s) may be accelerometer(s), light sensor(s), sound sensor(s), pressure sensor(s), temperature sensor(s), proximity sensor(s), infrared sensor(s), ultrasonic sensor(s), colour sensor(s), humidity sensor(s), tilt sensor(s), flow sensor(s), magnetic/Hall effect sensor(s), radiation sensor(s), lidar sensor(s), electrical current sensor(s), optical sensor(s), force/torque sensor(s), strain gauge(s), mechanical switch(es) etc.

The generated signals may be digital or analogue signals. A signal of the sensor unit may comprise the readings (signal values) of one or more sensor elements of the sensor unit. A signal “corresponding” to a data value may mean that a range of possible signal values is associated with that data value, e.g., to accommodate for deviations in the measurement process due to measurement errors and/or manufacturing inaccuracies. The signals may be uniquely identifiable to associate them with specific data values. The readings of a single sensor element may be associated with 0 when the signal value is below a specific value and with 1 when above or vice versa. Alternatively, the possible readings of a single sensor element may be divided into more than 2 ranges, e.g., 3, 4, 5, 6, 7, 8, 9, 10 or more ranges. Different ranges of signal values may be associated with different the data values. The ranges that associate with a specific data value may be static, i.e., predetermined or preprogrammed, or may be dynamic, e.g., dependent on one or more previous measurements, e.g., to accommodate for a degradation of the sensor unit and/or an energy source powering the sensor unit. A certain or minimum amount of movement by the dose control member may be needed for the sensor unit to generate signals that correspond to a different data value. The sensor unit and dose control member may be configured such that the data values corresponding to signals generated by the sensor unit change in equidistant intervals of movement (change in position) of the dose control member.

The electronic processing unit may comprise a processor unit configured to execute specific machine-readable code and/or an application specific integrated circuit (ASIC). The electronic processing unit may comprise a memory unit for storing at least the dose-related count. The electronic processing unit may further comprise a clock unit, e.g., a real-time clock. The electronic processing unit may be electrically connected with the sensor unit. The electronic processing unit may be configured to associate the signal values of the signals received from the sensor unit to the corresponding data values. Alternatively, the sensor unit may be configured to directly provide the corresponding data values instead of the signal values in the received signals. The sensor unit and the electronic processing unit may share a common integrated circuit (IC). The sensor unit may be configured to store the data values corresponding to the observed signals directly in the memory of the electronic processing unit. The electronic processing unit may be configured to be in a sleep state when receiving the signals from the sensor unit and may be configured to evaluate the received signals after waking from the sleep state. The electronic processing unit may be configured to store at least the most recent data value in the memory unit. The electronic processing unit may be configured to compare the first data value evaluated after waking from a sleep state with the last data value evaluated before entering the sleep state. The electronic processing unit may be configured to compare the first data value generated in a current dose set operation and/or dose dispense operation with a last data value generated in a directly previous dose set operation and/or dose dispense operation.

The electronic processing unit may be configured to disregard a pair of consecutive signals when the corresponding data value is the same.

The electronic processing unit may be configured to increase and/or decrease the dose-related count depending on the received signals. The dose-related count may be a data value representing an amount of medicament dispensed during the current dose dispense operation, a total amount of medicament dispensed from the currently installed medicament container or a total amount of medicament dispensed during the lifetime of the drug delivery device and/or the measurement system. The electronic processing unit may be configured to adapt more than one dose count depending on the received signals. Alternatively, the dose-related count may be a data value representing an amount of medicament selected in a dose set operation the user intends to dispense in a following dose dispense operation. As a further alternative, the dose-related count may be representative for an amount of medicament left in the medicament container.

The “repeating sequence” of the data values may be a sequence that repeats after a certain number N of elements such that the M^{th} element of the sequence is identical to the (M−N)^{th} element of the sequence. The certain number N of elements may be identical to the number of different data values. The repeating sequence may comprise every one of the different data values at least once, e.g., exactly once. Two data values are “neighbors” in the repeating sequence when one of the two data values is equal to the n^{th} data value in the repeating sequence, while the other of the two data values is equal to the n^{th}+1 data value. The repeating sequence may consist of a base sequence that is repeated multiple times, e.g., at least four times, e.g., at least ten times, e.g., at least twenty times, e.g., indefinitely.

The dose control member and the sensor unit may be configured such that if the sensor unit observed the dose control member continuously, e.g., with a very high sample rate, and the dose control member moved unidirectionally to increase the delivered or set dose or moved unidirectionally in a direction associated with an increase of the delivered or set dose, the predetermined repeating sequence would be produced, e.g., in the electronic processing unit.

Consecutive signals that correspond to the same data value may herein be regarded as the same signal and/or may be ignored.

According to further embodiments, the electronic processing unit may be configured to determine a movement direction of the dose control member based on the two consecutive signals and the predetermined repeating sequence at least when the two consecutive signals correlate. The two consecutive signals may comprise a leading signal and a trailing signal. The trailing signal may be generated after the leading signal. The leading signal may correspond to a first data value (that is one of the different data values). The trailing signal may correspond to a second data value (that is one of the different data values). The electronic processing unit may be configured to associate the two signals with a direction to increase a set or dispensed dose (forward direction) when the second data value is immediately following the first data value in the repeating sequence. The electronic processing unit may be configured to increase the dose related count, e.g., by one, when the two signals correspond to neighboring signals and a forward direction is determined. The electronic processing unit may be configured to associate the two signals with a direction to decrease a set or dispensed dose (backward direction) when the first data value is immediately following the second data value in the repeating sequence. The electronic processing unit may be configured to decrease the dose related count, e.g., by one, when the two signals correspond to neighboring signals and a backward direction is determined. According to further embodiments, the repeating sequence may be inversed, such that the association with the direction would be inverted as well. An increased accuracy of the dose measurement can be achieved, as for example the device may detect a backwards rotation and may therefore accommodate a change in dose count accordingly.

In other embodiments, the electronic processing unit may be configured to increase a sample rate of the sensor unit when the two consecutive signals do not correspond to neighbors in the repeating sequence of different signals. In a first mode of operation, a low sample rate may be chosen to only detect whether a movement of the dose control member has started. The electronic processing unit may be configured to switch the sensor unit to a high sample rate in a second mode of operation when the dose control member is determined to be in motion. The high sample rate may be associated with a sample rate at which the sensor unit may acquire corresponding data signals which reproduce the repeating series at least for any rate of movement (velocity and/or rotational speed) below an expected and/or typical maximum rate of movement. In a preferred embodiment the low sample rate may be associated with a sample rate at which the sensor unit may acquire corresponding data signals which reproduce at least every second of the repeating series at least for any rate of movement (velocity and/or rotational speed) below an expected and/or typical maximum rate of movement. The electronic processing unit may therefore be configured to increase the sample rate as soon as the movement of dose control member starts. Alternatively, or additionally, the electronic processing unit may be configured to set the sensor unit to a higher sample rate in a third mode of operation. The higher sample rate may be associated with a sample rate at which the sensor unit may acquire corresponding data signals which reproduce the repeating series at least for any rate of movement (velocity and/or rotational speed) below an extended rate of movement. The extended rate of movement may be at least as high, e.g., at least 1.3 times as high, e.g., at least 1.5 times as high, e.g., at least 2 times as high, as the expected and/or typical maximum rate of movement. This may increase an accuracy of the dose detection-especially, as the system may prevent an ambiguous situation in which due to accelerated movement the system would otherwise not be able to discern between a single transition and transition of full length of the repeating series plus one transition.

A partial sequence of the repeating sequence may span from a first (the first ever) occurrence of the second data value in the repeating sequence to a second (the second ever) occurrence of the second data value in the repeating sequence.

According to other embodiments, when the two consecutive signals are not neighbors in the repeating sequence, the electronic processing unit may be configured to increase the dose related count in relation (e.g., in proportion) to a first distance along the partial sequence from the first data value to the second occurrence of the second data value when a second distance along the partial sequence from the first occurrence of the second data value to the first data value is greater than a specific value, e.g., greater than 1. The electronic processing unit may be configured to increase the dose data in relation to a first distance along the repeating sequence from the first data value to the second data value when a second distance along an inverse of the repeating sequence from the first data value to the second data value is greater than a specific value, e.g., greater than 1. The electronic processing unit may be configured to increase the dose related count in relation to the first distance when the second distance is not bigger than the first distance. The electronic processing unit may be configured to increase the dose related count by the first distance. When assuming that a movement of the dose control member in the dose increase direction is more likely than in the backward direction, a reduction of error states can be achieved and a dose measurement accuracy can be increased. A “distance” along a sequence may be a count of changes of the values (transitions) along the sequence. The distance may be a difference in indices of the sequence. The distance from the N^{th} element of the sequence to the (N+m)^{th} element of the sequence may be m.

In other words, the electronic processing unit may be configured to determine (compute) a first distance between the first data value and the second value along the repeating sequence in a forward direction and to determine (compute) a second distance from the first data value and the second data value along the repeating sequence in a backwards direction. The electronic processing unit may be configured to increase the dose related count in relation (e.g., in proportion) to the first distance when the second distance is greater than a specific value, e.g., greater than 1. The processing unit may be configured to increase the dose related count in relation (e.g., in proportion) to an implied number of missing data values from the repeating sequence assuming a minimum distance along the repeating sequence from the first of the two consecutive signals to the second of the two consecutive signals and assuming the most likely dose control member direction of rotation being that which increases the dose delivered. By such means a reduction in dose measurement errors can be achieved and dose measurement accuracy can be increased.

A movement of the dose control member in relation to the housing opposite to the direction required to increase the delivered or set dose may at least be hindered. The drive mechanism of the drug delivery device may comprise at least one blocking member, e.g., a ratchet, that only allows movement that leads to an increase in dispensed medicament. The blocking member may provide a certain amount of leeway for a movement against a dose increase direction, while going beyond the leeway would only be possible by using excessive amounts of force and/or would lead to a (partial) destruction of the drug delivery device. This allows for the electronic processing unit to associate specific changes in detected position (change in data value corresponding to the received signals of the sensor unit) with movements in the forward direction.

Furthermore, the dose control member may be configured to rotate, e.g., in relation to and/or proportional to a dose set and/or dose delivered, and wherein the sensor unit is configured to generate the signals corresponding to different data values depending on angular positions of the dose control member. The sensor unit, e.g., at least the sensor elements of the sensor unit, may be arranged non-movable in relation to the housing and/or on the housing. Alternatively, the sensor unit, e.g., at least the sensor elements of the sensor unit, may be arranged on the dose control member or non-moveable in relation to the dose control member. This allows for an easy integration into existing designs. Alternatively, the dose control member may be configured and/or arranged to move linearly or in a helical fashion.

The predetermined repeating sequence may be associated with a Gray code sequence, e.g., a 2-bit Gray code sequence. A “Gray code sequence” may be a sequence of binary codes of a specific length where consecutive members of the sequence only differ in a single bit of the binary code. The Gray code sequence may comprise every possible binary bit combination of a given binary code length. The 2-bit Gray code sequence may consist of the sequence [00; 01; 11; 10]. Alternatively, the repeating sequence may be associated with a 3-bit Gray code sequence. Exemplarily, the 3-bit Gray code sequence may consist of the sequence [000; 001; 011; 111; 110; 100] or the sequence [000; 001; 011; 010; 110; 111; 101; 100]. Similarly, the repeating series could be associated with a Gray code of a higher bit-length. This allows for error detection and error correction.

According to further embodiments, e.g., when the sensor unit is fixed (non-moveable) in relation to the housing, the dose control member comprises a series of sensing regions, wherein neighboring sensing regions have different physical properties. According to alternative embodiments, the series of sensing regions may be arranged on the housing or an element fixed in relation to the housing, e.g., when the sensor unit is fixed in relation to the dose control member. “Having different physical properties” may herein mean that the sensing regions differ in value of at least one physical property that is observed by at least one sensor element of the sensor unit. The value of the physical property of the sensing region and/or a sensor response (e.g., sensor voltage or sensor current) created by a respective sensor element of the sensor unit when observing the respective sensing region, may herein be different by at least 10%, e.g., at least 11%, e.g., at least 100%, between any two neighboring regions. In comparison to sensing regions that have a continuously changing physical property along its surface, this does not require a high resolution in the sensor sensitivity. The series of sensing regions may consist of at least 2, e.g., at least 4, e.g., at least 6, e.g., at least 8, e.g., at least 10, e.g., at least 12 sensing regions.

The sensor unit may comprise at least two sensor elements observing the dose control member. The two sensor elements may be of the same kind, e.g., may be configured to observe (measure) the same physical property. Alternatively, the sensor elements may be configured to observe different physical properties, e.g., of the same sensing region. Multiple sensor regions may have the same physical properties, e.g., have values that differ by less than 10%, e.g., less than 5%. The sensing regions, e.g., at least the sensing regions having the same physical properties, may have the same size or different sizes. The “size” of the sensing region may herein refer to the extension of the sensing region according to a direction of movement, e.g., in relation to the sensor unit. At least two of the sensor elements may be arranged to observe different sensing regions at the same time. Two of the sensor elements may be arranged to observe different sensing regions at least at some time, and to observe the same sensing regions at least at different times. At least two of the sensor elements, e.g., the same two sensor elements, may be configured to observe the same sensing regions at different times. The sensor elements may be configured to observe the dose control member synchronously. The sensor elements may sample at the same time(s) and may have the same sample rate. The sensor elements may be arranged to observe the dose control member out of phase. According to alternative embodiments, the two sensor elements may be configured to never observe the same sensing regions. The sensing regions may form a first series of sensing regions to be observed by a first sensor element and a second series of sensing regions to be observed by a second sensor element. The sensing elements of the second series may be larger, e.g., twice as large, as the sensing elements of the first series.

A full rotation (360°) of the dose control member may correspond to a multiple of the base sequence of the repeating sequence. It may for example correspond to a 1 times, 2 times, 3 times, 4 times, 5 times, 6 times, 7 times or 8 times repetition of the base sequence, but other configurations are also possible.

According to other embodiments, the dose control member comprises a ring-shaped portion and the sensing regions are arranged along its circumference. At least two of the sensor elements, e.g., any two of (a subset of) the sensor elements, may be arranged such that when one of the sensor elements is directed towards a transition between two sensing regions the other sensor element is not directed towards a transition between two sensing regions, e.g., directed toward the center of another sensing region. This allows for an easy implementation of the repeating sequence.

Additionally, a drug delivery device is proposed, comprising a measurement system of any one of the previous claims or configured to be coupled with such a measurement system, e.g., releasably coupled. A housing of the drug delivery device may comprise an electronic and/or mechanical interface. The measurement system may comprise a separate housing comprising an electronic and/or mechanical interface configured to be connected with the interface of the housing. The interfaces may comprise means to releasably lock the measurement system to the drug delivery device. The drug delivery device may be an injection device. The drug delivery device may be a needle-based injection device. The drug delivery device may be a pen-type device. Pen-type devices comprise a form similar to a pen, e.g., a fountain pen, thereby allowing an easy handling of the medical device by users such as for self-administration. The drug delivery device may be a handheld device.

The drug delivery device may comprise at least one container filled with medicament or a receptacle for receiving such a container. The container may comprise medicament in an amount sufficient for a plurality of doses to be dispensed by the device.

In case the measurement system is integrated into the device and in case the device is a drug delivery device, the device can preferably be used with a plurality of containers. In other words, the device may be a reusable device. Of course, the measurement system as an add-on module could also be used with a reusable medical device. The user may just have to replace an emptied container in the receptacle with a new one and re-connect the receptacle to a drive mechanism of the device.

The drug delivery device may be a variable dose device, where the size of the dose to be delivered can be set within limits defined by the mechanism of the device.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 11 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively, or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g., a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B11) human insulin; Des(B27) human insulin and Des(B11) human insulin.

Examples of insulin derivatives are, for example, B29-N-myristoyl-des(B11) human insulin, Lys(B29) (N-tetradecanoyl)-des(B11) human insulin (insulin detemir, Levemir®); B29-N-palmitoyl-des(B11) human insulin; B29-N-myristoyl human insulin; B29-N-palmitoyl human insulin; B28-N-myristoyl LysB28ProB29 human insulin; B28-N-palmitoyl-LysB28ProB29 human insulin; B11-N-myristoyl-ThrB29LysB11 human insulin; B11-N-palmitoyl-ThrB29LysB11 human insulin; B29-N—(N-palmitoyl-gamma-glutamyl)-des(B11) human insulin, B29-N-omega-carboxypentadecanoyl-gamma-L-glutamyl-des(B11) human insulin (insulin degludec, Tresiba®); B29-N—(N-lithocholyl-gamma-glutamyl)-des(B11) human insulin; B29-N-(ω-carboxyheptadecanoyl)-des(B11) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-1191 March-701, MAR709, ZP-2929, ZP-1122, ZP-DI-70, TT-401 (Pegapamodtide), BHM-034. MOD-6011, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F (ab) and F (ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full-length antibody polypeptide, although the term is not limited to such cleaved fragments. Antibody fragments that are useful in the present invention include, for example, Fab fragments, F (ab′) 2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

Furthermore, a method for measuring the dose dispensed and/or dose set of a drug delivery device is proposed, comprising: intermittently observing the position and/or orientation of a dose control member that moves in accordance with a dose dispense operation and/or a dose set operation; generating signals, each corresponding to one of at least four different data values, depending on a position and/or orientation of the dose control member; adapting a dose-related count depending on the generated signals; and determining whether two consecutive of the generated signals correspond to neighboring data values in a predetermined repeating sequence of different data values.

Adapting the dose-related count may comprise determining a movement direction of the dose control member. Adapting the dose-related count may comprise increasing the-dose related count when a movement of the dose control member in a dose-increase direction (forwards) is determined. Adapting the dose-related count may comprise decreasing the dose-related count when a movement of the dose control member in a dose-decrease direction (backwards) is determined. When the two consecutive signals are not neighbors in the repeating sequence, the dose related count may be increased in relation to a distance along the partial sequence from a first data value corresponding to a first of the generated signals to a later occurrence of a second data value corresponding to a second of the generated signals when a distance along the partial sequence from an earlier occurrence of the second data value to the first data value is greater than a specific value, e.g., greater than 1. In other words, the dose related count may be increased in relation to an implied number of missing data values from the repeating sequence assuming a minimum distance along the repeating sequence from the first of the two consecutive signals to the second of the two consecutive signals and assuming the most likely dose control member direction of rotation being that which increases the dose delivered.

The method may further comprise storing the dose-related count, e.g., in a memory unit of the measurement system for the drug delivery device.

In an embodiment, a method for measuring the dose dispensed and/or dose set with a drug delivery device according to one of the embodiments of the present disclosure is provided.

Furthermore, machine-readable code is proposed that when executed in a electronic processing unit of a measurement system causes the system to behave and act like a before mentioned measurement system. The machine-readable code may be provided in form of an upgrade to already existing machine-readable code present in the measurement system.

Additionally, a data-storage medium, e.g., a compact disc, a flash drive, a memory card, a cloud storage or a data stream, comprising the before mentioned machine-readable code or machine-readable code to reproduce the before mentioned machine-readable code in the measurement system (update), is proposed.

### BRIEF DESCRIPTION OF THE DRAWINGS

FIG. 1 shows a schematic view of a drug delivery device comprising a measurement system.

FIG. 2 shows a schematic sectional view along Marker A in FIG. 1.

FIG. 3 shows a partial sequence of a repeating sequence according to a Gray code sequence.

### DESCRIPTION OF EXEMPLARY EMBODIMENTS

In the figures, identical elements, identically acting elements or elements of the same kind may be provided with the same reference numerals.

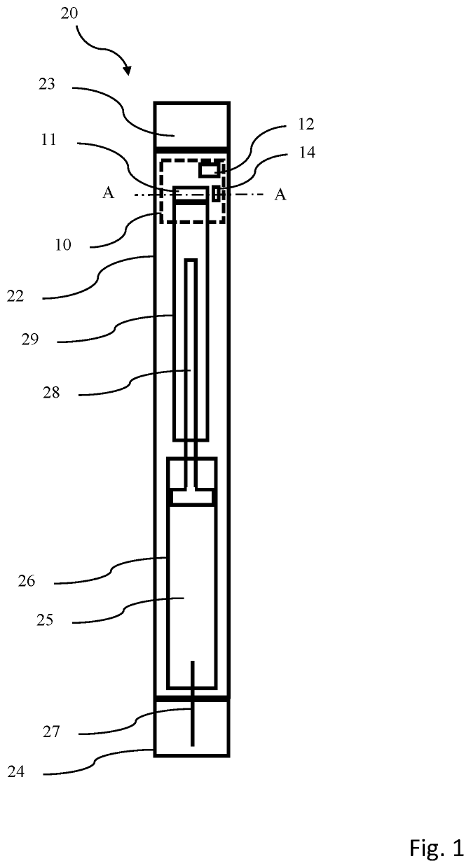

FIG. 1 shows a drug delivery device 20. The drug delivery device 20 comprises an electronic measurement system (measurement system) 10 for the drug delivery device 20. The measurement system 10 comprising an electronic processing unit 12. The measurement system 10 comprising a sensor unit 14. The measurement system 10 is configured to measure an amount of drug dispensed from the drug delivery device 20.

The electronic processing unit 12 may comprise a memory unit for storing the measurement amounts of dispended drug. The electronic processing unit 12 may additionally comprise a communication unit 14 for transmitting measurement amounts of drug and other data like times at which the drug was administered to an external device.

The drug delivery device 20 is a needle-based injection device (also called NIS-needle-based injection system). The drug delivery device 20 is a pen-type device. The drug delivery device 20 comprises an elongated housing 22. The drug delivery device 20 comprises a receptacle 26 for receiving a container 25. The container 25 is filled with a medicament. The container 25 may be changed for a different one by partly disassembling the drug delivery device 20, e.g., an empty container may be changed for a full one. The drug delivery device 20 comprises a needle 27 at a first end that is fluidly connected with the container 25. The drug delivery device 20 comprises a cap 24 that may be mechanically connected to the housing 22 and that is covering the needle 27 when connected with the housing 22. The drug delivery device 20 comprises a dose dial 23 rotatably connected to the housing 22 at a second end opposite from the needle 27. A dose of medicament to be ejected from the drug delivery device 20 (and injected into the human body) may be selected by rotating the dose dial 23. The dose dial 23 also functions as a release to initiate dispense of the medicament upon a press along the cylinder axis. Alternatively, a separate button to be manually pressed by the user or a needle shield that is retracted upon insertion of the needle 27 into the human body and thereby triggers the release may be provided to fulfill that function. The drug delivery device 20 comprises a drive unit 29 and a plunger 28. The drive unit 29 drives the plunger 28 for a distance specific to the set dose into the container 25.

The electronic measurement system 10 comprises a dose control member 11. The dose control member 11 is configured to move in accordance with a dose dispense operation in relation to the housing 22 of the drug delivery device 20. According to other embodiments, the dose control member is configured to move in accordance with a dose set operation or both operations.

The electronic measurement system 10 comprises a sensor unit 14. The sensor unit 14 is electrically connected to the electronic processing unit 12. The sensor unit 14 is configured to observe a position of the dose control member 11 intermittently. The sensor unit 14 is configured to generate signals, each corresponding to one of four different data values A, B, C or D, depending on a position of the dose control member 11. The electronic measurement system 10 further comprises an energy source, e.g., an electric battery, to power the electronic processing unit 12 and the sensor unit 14.

For driving the plunger 28, the drive unit 29 creates a rotary movement that is translated into a linear movement of the plunger 28. The dose control member 11 is configured to rotate an amount proportional to an amount of linear movement of the plunger 28.

The electronic processing unit 12 is configured to receive the signals of the sensor unit 14, to adapt a dose-related count depending on the received signals. The electronic processing unit 12 is configured to determine whether two consecutive data values of the received signals are neighbors in a predetermined repeating sequence [ . . . , A, B, C, D, A, B, C, D, . . . ] of the four different data values A, B, C and D.

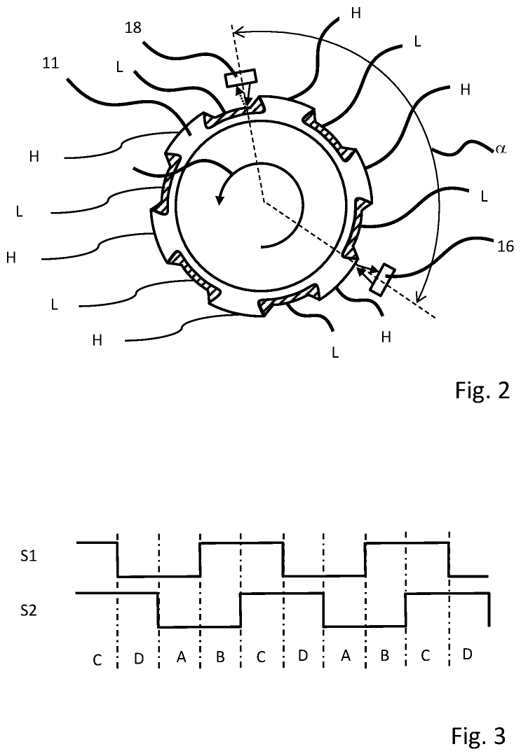

The sensor unit 14 comprises two sensor elements 16, 18 observing the dose control member 11 (compare FIG. 2). The sensor elements 16, 18 are optical sensors. The sensor elements 16, 18 are configured to observe a reflectivity of the dose control member 11. The sensor elements 16, 18 are configured to emit light (in this case infrared light) towards the dose control member 11 and to detect how much light is received back in the sensor element 16, 18.

The dose control member 11 comprises a series of sensing regions L, G, wherein neighboring sensing regions L, H have different physical properties. The series of sensing regions comprises 12 sensing regions L, H. The sensing regions L, H each occupy 30° of the dose control member. Half of the sensing regions L have a low reflectivity (for IR light). The other half of the sensing regions H have a high reflectivity (for IR light). The sensing regions L, H with low and high reflectivity are arranged alternatingly. Other embodiments may use more or fewer sensing regions. Using more sensing regions would allow for a higher resolution, while fewer sensing regions would allow for lower sample rates and lower energy consumption. The sensing regions L that have a lower value of the physical property are made from a different material than the sensing regions H that have a higher value of the physical property. The sensing regions L that have a low reflectivity are recessed to increase the distance between the sensor and the sensing regions L that have a low reflectivity, thereby further decreasing the signal output by the sensor when observing a low reflectivity region. This improves the contrast between regions and the corresponding difference in signal output from the sensors when observing the two regions. A first kind of the sensing regions H (the ones with the high value of the physical property in this case) may be constituted by a base material of the dose control member 11. The other sensing regions L may be formed by an additive process, e.g., adding material to the base material in between the first kind of sensing regions H, e.g., by injection molding, or by formfitting and/or adhering one or more additional parts.

The dose control member 11 is an essential part of the drive unit 29 and transfers energy to the plunger 28. The dose control member 11 comprises a ring-shaped portion. The ring-shaped portion is arranged on a proximal end of the dose control member 11. The sensing regions L, H are arranged along a circumference of the ring-shaped portion. The sensing regions are arranged on a radially outwards surface of the ring-shaped portion. The sensor elements 16, 18 are arranged distanced to the ring-shaped portion in a radially outward direction from the dose control member 11. The sensor elements 16, 18 are fixed in relation to the housing 22. The sensor elements 16, 18 are arranged such that when one of the sensor elements 16, 18 is directed towards a transition between two sensing regions L, H, the other sensor element 16, 18 is not directed towards a transition between two sensing regions (L, H). It is directed toward the center of another sensing region L, H. The sensor elements 16, 18 are arranged to observe different sensing regions L, H at the same time (at any time), and to observe the same sensing regions L, H at different times. The sensing regions L, H each occupy a same amount of space (in this case 30°) of the dose control member. The sensor elements 16, 18 are distanced angularly in relation to a center of the ring-shaped portion of the dose control member by an angle α. The angle α is 135°.

During operation of the device, when dispensing (or dialling according to other embodiments), a pair of sensors are mechanically coupled to an encoded component (Encoder Ring).

The sensor unit 12 is configured to generate the signals corresponding to the different data values A, B, C or D depending on angular positions of the dose control member 11. The predetermined repeating sequence [ . . . , A, B, C, D, A, B, C, D, . . . ] is associated with a 2-bit Gray code (compare FIG. 3). According to the physical properties of the respectively observed sensing region L, H a first of the sensor elements 16 generates a first sensor signal S1 with the value 0 (low value of the physical property) or 1 (high value of the physical property). According to the physical properties of the respectively observed sensing region L, H a second of the sensor elements 18 generates a second sensor signal S2 with the value 0 (low value of the physical property) or 1 (high value of the physical property). The dose control member 11 and the sensor unit 14 are configured such that when the sensor unit 14 observed the dose control member 11 continuously and the dose control member 11 moved unidirectionally in a direction R to increase the delivered or set dose, the predetermined repeating sequence [ . . . , A, B, C, D, A, B, C, D, . . . ] would be produced.

In the case of an optical sensor, and dependent upon the last dispensed dose, each sensor could be pointing at either a black or white region. Nominally, the centre of each sensor is positioned a certain angle away from a transition edge. The sensor response whilst pointing at a white region is defined as a binary 1, and a binary 0 whilst pointing at a black region. Configurations, whereby the sensors are nominally pointing at one of two states and transitions occur between these states, are applicable to other sensor technologies, as listed above.

In a position of the dose control member 11 in which both sensor elements 16, 18 are directed towards a sensing region L with a low value of the physical property (IR reflectivity), the sensor unit 14 would produce a signal “00” from the sensor unit 14 that is associated with a first of the different data values A. In a position of the dose control member 11 in which the first sensor element 16 is directed towards a sensing region H with a high value of the physical property, while the second sensor element 18 is directed towards a sensing region L with low value of the physical property, the sensor unit 14 would produce a signal “10” from the sensor unit 14 that is associated with a second of the different data values B. In a position of the dose control member 11 in which both sensor elements 16, 18 are directed towards a sensing region H with a high value of the physical property, the sensor unit 14 would produce a signal “11” from the sensor unit 14 that is associated with a third of the different data values C. In a position of the dose control member 11 in which the first sensor element 16 is directed towards a sensing region L with a low value of the physical property, while the second sensor element 18 is directed towards a sensing region H with high value of the physical property, the sensor unit 14 would produce a signal “10” from the sensor unit 14 that is associated with a fourth of the different data values D. The first of the data values A is followed by the second of the data values B in the repeating sequence [ . . . , A, B, C, D, A, B, C, D, . . . ]. The second of the data values B is followed by the third of the data values C in the repeating sequence [ . . . , A, B, C, D, A, B, C, D, . . . ]. The third of the data values C is followed by the fourth of the data values D in the repeating sequence [ . . . , A, B, C, D, A, B, C, D, . . . ]. The fourth of the data values D is followed by the first of the data values A in the repeating sequence [ . . . , A, B, C, D, A, B, C, D, . . . ]. The position given in FIG. 2 herein correlates with the signal “10” from the sensor unit 14 and corresponds to the second of the different data values B. The different data values A, B, C and D are identical to the respective signals of the sensor unit 14.

A user of the drug delivery device 20 may initiate a use of the device by using the dose dial 23 to select a dose (amount of drug) to be dispensed in a set dose operation. During the set dose operation, the sensor unit 14 is deactivated and does not provide signals for determining data values representing positions of the dose control member 11. The electronic control 12 unit may be deactivated or in a sleep mode in this stage.

When the user initiates the dose dispense operation by pressing on the dose dial 23 or by a different mechanism, in a first stage, e.g., by closing a contact upon reaching a first pressing depth of the dose dial 23, the sensor unit 14 is activated and set to a first sample rate. The electronic processing unit 12 may also be activated to evaluate the signals of the sensor unit. The dose related count is set to zero. The electronic processing unit 12 is configured to retrieve a saved data value corresponding to a last received signal of the sensor unit 14 of a previous dose dispense operation from memory to allow for a comparison with the newly generated signals. Alternatively, the first data value for comparison may be a factory preset. Also, the sensor unit may be activated during a medicament container replacement to establish and acquire a reset position.

The drive unit 29 comprises a lock mechanism to prevent a movement of the plunger 28 and the dose control member 11 (amongst possible other parts of the drive unit 29). In a second stage, e.g., after a second pressing depth of the dose dial (later/deeper than the first pressing depth) is reached, the drive unit 29 is unlocked to dispense the drug by deactivating the lock mechanism at least for the direction R to increase the delivered dose. A movement of the dose control member 11 in relation to the housing opposite to the direction R to increase the delivered dose is at least hindered by the lock mechanism in any stage.

When any one of the sensor elements changes from 0 to 1, or 1 to 0, i.e., a transition occurs, it is interpreted that one unit has been dispensed, but may also be interpreted as a multiple of a single unit, or a fraction of a single unit, depending on the configuration of the system. Due to the implementation of Gray coding, it is possible to determine whether the unit will increase or decrease the overall dose count. Furthermore, the mechanism is designed in such a way that the dose will only increase in normal operation. Any consecutive transitions that indicate a decrease in overall dose count suggests a mechanical and/or electrical failure, or momentary oscillations in a reverse direction, so-called ‘jitter’.

The electronic processing unit 12 is configured to increase a sample rate of the sensor unit 14 when the two consecutive signals do not correspond to neighbors in the repeating sequence [ . . . , A, B, C, D, A, B, C, D, . . . ] of different signals A, B, C or D. The electronic processing unit 12 is configured to increase the sample rate of the sensor unit 14 at least after a first movement (any change of the data value A, B, C or D corresponding to the received signals from the sensor unit 14) of the dose control member 11 is detected. Especially when the dose dial 23 is pressed very hard, the first couple of transitions of different data values A, B, C or D in the repeating sequence [ . . . , A, B, C, D, A, B, C, D, . . . ] may be missed due to a delay in activation of the sensor unit 14.

When an amount of medicament according to the set dose is dispensed, the drive mechanism reaches an end-position in which forward movement in direction R is again blocked by the lock mechanism. Especially at the end of the dose dispense operation, a momentary oscillation (jitter movement) of parts of the drive unit 29 and/or the dose control member 11, e.g., due to flexibility in the part and mechanism, may occur leading to the sensor unit 14 providing signals switching back and forth between two data values. The electronic processing unit 12 is configured to determine a movement direction of the dose control member 11 based on the two consecutive signals and the predetermined repeating sequence [ . . . , A, B, C, D, A, B, C, D, . . . ]. When the electronic processing unit 12 determines the consecutive signals to correlate with two neighboring data values (A, B), (B, C), (C, D), (D, A) in the forward direction R, the electronic processing unit 12 increases the dose related count by one. When the electronic processing unit 12 determines the consecutive signals to correlate with two neighboring data values (B, A), (C, B), (D, C), (A, D) in the opposite of the forward direction R, the electronic processing unit 12 decreases the dose related count by one. When the electronic processing unit 12 determines the consecutive signals to repeatedly correlate with two neighboring data values (B, A), (C, B), (D, C), (A, D) in the opposite of the forward direction R, the electronic processing unit 12 throws an error, as such movement should not be possible within the restraints of the drug delivery device 20 without a critical device failure. An error message or warning may be provided to the user acoustically and/or visually.

When dispensing at higher rates that approach and exceed the sampling rate of the measurement system (encoder system), it is possible that consecutively measured transitions do not correspond to consecutive Gray code transitions. In the example of an optical sensor system, an expected set of transitions would be “11”→“01”→“00”→“10”. However, if the system goes from “11” to “00”, it is not clear whether the transition is forwards or backwards i.e., “11”→“01”→“00” (forwards) or “11”→“10”→“00” (backwards). This is referred to as a double transition and may be that either the “01” or “10” position has effectively been skipped hence it is not possible to determine the direction.

The two consecutive signals comprise a leading signal corresponding to a first data value A, B, C or D and a trailing signal corresponding to a second data value C, D, A or B. A partial sequence spans from a first occurrence of the second data value C, D, A or B in the repeating sequence [ . . . , A, B, C, D, A, B, C, D, . . . ] to the second occurrence of the second data value C, D, A or B in the repeating sequence [ . . . , A, B, C, D, A, B, C, D, . . . ]. E.g., when the second data value is C, the partial sequence would be [C, D, A, B, C].

The electronic processing unit is configured to determine a first distance along the repeating sequence from the first data value to the second data value in a forwards direction. This can be seen as determining a distance (first distance) along the partial sequence from the first data value A, B, C, D to the second occurrence of the second data value C, D, A or B. The electronic processing unit is further configured to determine a second distance along the repeating sequence from the first data value to the second data value in a backwards direction. This can be seen as determining a distance (second distance) along the partial sequence from the first occurrence of the second data value C, D, A or B to the first data value A, B, C or D.

When the two consecutive signals are not corresponding to neighbors in the repeating sequence [ . . . , A, B, C, D, A, B, C, D, . . . ], the electronic processing unit 12 is configured to increase the dose related count in relation to first distance (in the case of only four different data values always by 2) when a second distance is greater than a specific value, in this case greater than 1.

A double transition is always interpreted as forwards i.e., increasing the overall dose count (dose related count). This is because it is physically impossible to drive the mechanism backwards by more than one transition without some fault or abuse condition. In effect, the embedded software is written taking into account the physical characteristics of the overall system. This ensures that double transitions do not trigger unnecessary fault conditions whilst improving dose record accuracy.

For example, the system is being dispensed quickly and a double transition is detected. The previous dose count was 20 units, but using the system architecture described herein, the double transition adds 2 units to the dose count resulting in a dose of 22 units. The next transition is as expected and increases the dose count by one to 23 units.

The electronic processing unit may determine that the dose dispense operation has come to an end, when the dose button is released. Alternatively, the electronic processing unit may determine that the dose dispense operation ends, when the electronic processing unit 12 determines that the drive unit 29 has come to a stop due to a lack of change in the data values corresponding to the signals of the sensor unit 14. The electronic processing unit 12 is configured to store the measured dose related count to memory, e.g., together with a timestamp. Furthermore, the data value A, B, C or D corresponding to the latest signal of the sensor unit 14 is committed to memory.

With this procedure, an approach to identifying and accounting for non-consecutive Gray code transitions detected by a pen injection system utilising a pair of sensors is achieved. The method accounts for the physical characteristics of the system, improves dose record accuracy and reduces unnecessary fault conditions being identified.

Due to the fact that the embodiment switches to the faster sample rate when any transition is detected (including a double transition) it is configured such that most physically possible accelerations do not lead to a missed transition when the slow sample rate is active (i.e., a triple or greater transition event). Furthermore, the mechanical velocity of the system needs to exceed twice the fast sample rate in order for there to be triple or greater transitions when the fast sample rate is active. The sample rates are so chosen in order that such a speed is not physically possible with the mechanism so that the sensor system is configured to be robust against losing count under high speed injection events.

In other embodiments, there could e.g., be 8 different data values. An electronic processing unit may then be configured to increase the dose related count in relation to the calculated number of missed transitions (1, 2, 3, 4 or 5) increasing the dose count by (2, 3, 4, 5 or 6). A calculated 6th missed transition would then be interpreted as a single reverse transition and the dose count would be reduced by one. Furthermore, the electronic processing unit may be configured to record an error when the number of missed transitions is above a set threshold (for example 2, 3, 4, or 5).

In alternative embodiments, the sensor unit could only have a single sensor element, but the sensing regions have a number of different values or value ranges in the observed physical property while the sensor element is configured to individually distinguish these different values or value ranges to each correspond to a different data value.

In further embodiments the sensor unit may comprise three sensor elements instead of the proposed two, arranged at 0°, 130° and 260° to produce a 3-bit Gray code (or at least Gray-like code).

Also in alternative embodiments, the sensing regions may be arranged on an axial face of the ring, e.g., remote from the needle and/or on a proximal end of the dose control member, and the sensor elements may be arranged distal to the dose control member in an axial direction, e.g., remote from the needle or proximally.

### REFERENCE NUMERALS

10 measurement system
11 dose control member
12 electronic processing unit
14 sensor unit
16 sensor element
18 sensor element
20 drug delivery device
22 housing
23 dose dial
24 cap
25 container
26 receptacle
27 needle
28 plunger
29 drive unit
A data value
B data value
c data value
D data value
H sensing region
L sensing region
R direction
S1 sensor signal
S2 sensor signal

1. Measurement system for a drug delivery device (20), comprising
a dose control member (11) configured to move in accordance with a dose dispense operation and/or a dose set operation, e.g., in relation to a housing (22) of the drug delivery device (20),
a sensor unit (14) for observing a position of the dose control member (11) intermittently, the sensor unit being configured to generate signals, wherein each signal corresponds to one of at least four different data values (A, B, C or D), depending on a position of the dose control member (11), and
an electronic processing unit (12) configured to receive the signals of the sensor unit (14), to adapt a dose-related count depending on the received signals and to determine whether two consecutive of the received signals correspond to data values that are neighbors in a predetermined repeating sequence of the different data values (A, B, C and D).

2. The measurement system of claim 1, wherein the dose control member (11) and the sensor unit (14) are configured such that when the sensor unit (14) observes the dose control member (11) continuously and the dose control member (11) moves unidirectionally in a direction (R) to increase the delivered or set dose, the predetermined repeating sequence is produced.

3. The measurement system of any one of the preceding claims, wherein the electronic processing unit (12) is configured to determine a movement direction of the dose control member (11) based on the two consecutive signals and the predetermined repeating sequence.

4. The measurement system of any one of the preceding claims, wherein the electronic processing unit (12) is configured to increase a sample rate of the sensor unit (14) when the two consecutive signals do not correspond to data values that are neighbors in the repeating sequence of different signals.

5. The measurement system of any one of the preceding claims, wherein the two consecutive signals comprise a leading signal corresponding to a first data value (A, B, C, D) and a trailing signal corresponding to a second data value (A, B, C, D), wherein, when the two consecutive signals do not correspond to neighbors in the repeating sequence, the electronic processing unit (12) is configured to determine a first distance from the first data value (A, B, C, D) to the second data value (A, B, C, D) along the repeating sequence in a forwards direction and to determine a second distance along the repeating sequence from the first data value (A, B, C, D) to the second data value (A, B, C, D) in a backwards direction, and wherein the electronic processing unit (12) is configured to increase the dose related count in relation to the first distance when the second distance is greater than a specific value, e.g., greater than 1.

6. The measurement system of any one of the preceding claims, wherein a movement of the dose control member (11) in relation to the housing (22) opposite to the direction (R) to increase the delivered or set dose is at least hindered.

7. The measurement system of any one of the preceding claims, wherein the dose control member (11) is configured to rotate and wherein the sensor unit (14) is configured to generate the signals corresponding to the different data values (A, B, C or D) depending on angular positions of the dose control member (11).

8. The measurement system of any one of the preceding claims, wherein the predetermined repeating sequence is associated with a Gray code, e.g., a 2-bit Gray code.

9. The measurement system of any one of the preceding claims, wherein the dose control member (11) comprises a series of sensing regions (L, H), wherein neighboring sensing regions (L, H) have different physical properties.

10. The measurement system of any one of the preceding claims, wherein the sensor unit comprises at least two sensor elements (16, 18) observing the dose control member (11).

11. The measurement system of claims 9 and 10, wherein at least two of the sensor elements (16, 18) are arranged to observe different sensing regions (L, H) at least at some time, and to observe the same sensing regions (L, H) at least at different times.

12. The measurement system of claim 11, wherein the dose control member (11) comprises a ring-shaped portion and the sensing regions (L, H) are arranged along its circumference and the sensor elements (16, 18) are arranged such that when one of the sensor elements (16, 18) is directed towards a transition between two sensing regions (L, H) the other sensor element (16, 18) is not directed towards a transition between two sensing regions (L, H), e.g., directed toward the center of another sensing region (L, H).

13. Drug delivery device comprising a measurement system (10) of any one of the previous claims or configured to be coupled with such a measurement system, e.g., releasably coupled.

14. Drug delivery device of claim 13, comprising at least one container (25) filled with medicament or a receptacle (26) for receiving such a container (25).

15. Method for measuring the dose dispensed and/or dose set of a drug delivery device, e.g., the drug delivery device (20) of claim 13 or 14, comprising:
intermittently observing the position and/or orientation of a dose control member (11) that moves in accordance with a dose dispense operation and/or a dose set operation
generating signals, each corresponding to one of at least four different data values (A, B, C or D), depending on a position and/or orientation of the dose control member (11),
adapting a dose-related count depending on the generated signals, and
determining whether two consecutive of the generated signals correspond to neighboring data values in a predetermined repeating sequence of the different data values.
